(19) United States
(12) Patent Application Publication
(10) Pub. No.: US 2026/0033784 A1
(43) Pub. Date: Feb. 5, 2026

(54) APPARATUS AND METHOD FOR FILTERING A POTENTIAL SIGNAL USING CATHETER VELOCITY DURING ELECTRO-ANATOMICAL MAPPING

(71) Applicant: Anumana, Inc., Cambridge, MA (US)

(72) Inventors: Animesh Agarwal, San Mateo, CA (US); Rohit Jain, Danville, CA (US)

(73) Assignee: Anumana, Inc., Cambridge, MA (US)

(21) Appl. No.: 18/788,704

(22) Filed: Jul. 30, 2024

(51) Int. Cl. A61B 5/00; A61B 5/06; A61B 5/287; A61B 5/346; A61B 5/367; A61M 25/01

(52) U.S. Cl. CPC A61B 5/721; A61B 5/066; A61B 5/287; A61B 5/346; A61B 5/367; A61M 2025/0166

(57) ABSTRACT

An apparatus and method for filtering a potential signal using catheter velocity during electro-anatomical mapping. The apparatus includes at least a processor and a memory communicatively connected to the at least a processor. The memory instructs the processor to derive at least a velocity signal from the at least a position signal. The memory instructs the processor to identify, using the at least a velocity signal, a local minimum velocity. The memory instructs the processor to process, using a motion filter, the at least a position signal as a function of the at least a minimum velocity time and the at least a minimum velocity position.

### FIELD OF THE INVENTION

The present invention generally relates to the field of signal analysis. In particular, the present invention is directed to an apparatus and a method for filtering a potential signal using catheter velocity during electro-anatomical mapping.

### BACKGROUND

Electro-anatomical (EA) mapping involves the collection of electrical signals from within the heart to create detailed maps of the heart's electrical activity. This process is important for diagnosing and treating various cardiac conditions. The quality of the acquired signals can be significantly affected by the motion of the heart, which includes both cardiac and respiratory movements. These motions can introduce noise and artifacts into the data, complicating the interpretation and accuracy of the maps.

Current methods for assessing signal quality during EA mapping often fail to account for the complex and asynchronous nature of cardiac and respiratory motions. These motions can vary significantly across different regions of the heart and at different times, leading to inconsistencies in the data.

### SUMMARY OF THE DISCLOSURE

In an aspect, an apparatus for filtering a potential signal using catheter velocity during electro-anatomical mapping includes at least a processor and a memory communicatively connected to the at least a processor. The memory contains instructions configuring the processor to derive at least a velocity signal from the at least a position signal, identify, using the at least a velocity signal, a local minimum velocity and corresponding at least a minimum velocity time and at least a minimum velocity position, and process, using a motion filter, the at least a position signal as a function of at least a minimum velocity time and at least a minimum velocity position.

In another aspect, a method for filtering a potential signal using catheter velocity during electro-anatomical mapping includes deriving at least a velocity signal from the at least a position signal, identifying, using the at least a velocity signal, a local minimum velocity and corresponding at least a minimum velocity time and at least a minimum velocity position, and processing, using a motion filter, the at least a position signal as a function of the at least a minimum velocity time and the at least a minimum velocity position.

These and other aspects and features of non-limiting embodiments of the present invention will become apparent to those skilled in the art upon review of the following description of specific non-limiting embodiments of the invention in conjunction with the accompanying drawings.

### BRIEF DESCRIPTION OF THE DRAWINGS

For the purpose of illustrating the invention, the drawings show aspects of one or more embodiments of the invention. However, it should be understood that the present invention is not limited to the precise arrangements and instrumentalities shown in the drawings, wherein:

FIG. 1 is a block diagram of an apparatus for filtering a potential signal using catheter velocity during electro-anatomical mapping;

FIG. 2 is an illustration of a graph of an RA-1 file cardiac motion 4 second window;

FIG. 3 is an illustration of a graph of an RA-1 file respiratory motion 13 second window;

FIG. 4 is an illustration of a graph of an RA-1 file respiratory motion 13 second window typical catheter location movement;

FIG. 5 is an illustration of a graph of an RV-1 file cardiac motion 4 second window;

FIG. 6 is an illustration of a graph of an RV-1 file respiratory motion 13 second window;

FIG. 7 is an illustration of a graph of an RV-1 file all motion 13 second window;

FIG. 8A is an illustration of a graph of an RV-1 file cardiac motion 13 second window showing the smoothed curve;

FIG. 8B is an illustration of a graph of an RV-1 file cardiac motion 13 second window showing the diastolic envelope;

FIG. 9 is an illustration of a graph of an RV-1 file cardiac motion 95 second window with smoothed Y curve;

FIG. 10 is a block diagram of an exemplary method for filtering a potential signal using catheter velocity during electro-anatomical mapping;

FIG. 11 is a block diagram of a computing system that can be used to implement any one or more of the methodologies disclosed herein and any one or more portions thereof.

The drawings are not necessarily to scale and may be illustrated by phantom lines, diagrammatic representations and fragmentary views. In certain instances, details that are not necessary for an understanding of the embodiments or that render other details difficult to perceive may have been omitted.

### DETAILED DESCRIPTION

At a high level, aspects of the present disclosure are directed to apparatus and methods for filtering a potential signal using catheter velocity during electro-anatomical mapping. The apparatus includes at least a catheter configured for intracardiac use, at least a localization system configured to detect at least a position signal as a function of a location of the at least a catheter, and at least a computing device comprised of a processor and a memory communicatively connected to the processor. The processor derives at least a velocity signal from the at least a position signal. The memory instructs the processor to identify, using the at least a velocity signal, a local minimum velocity and corresponding at least a minimum velocity time and at least a minimum velocity position. The processor processes, using a motion filter, the at least a position signal as a function of the at least a minimum velocity time and the at least a minimum velocity position.

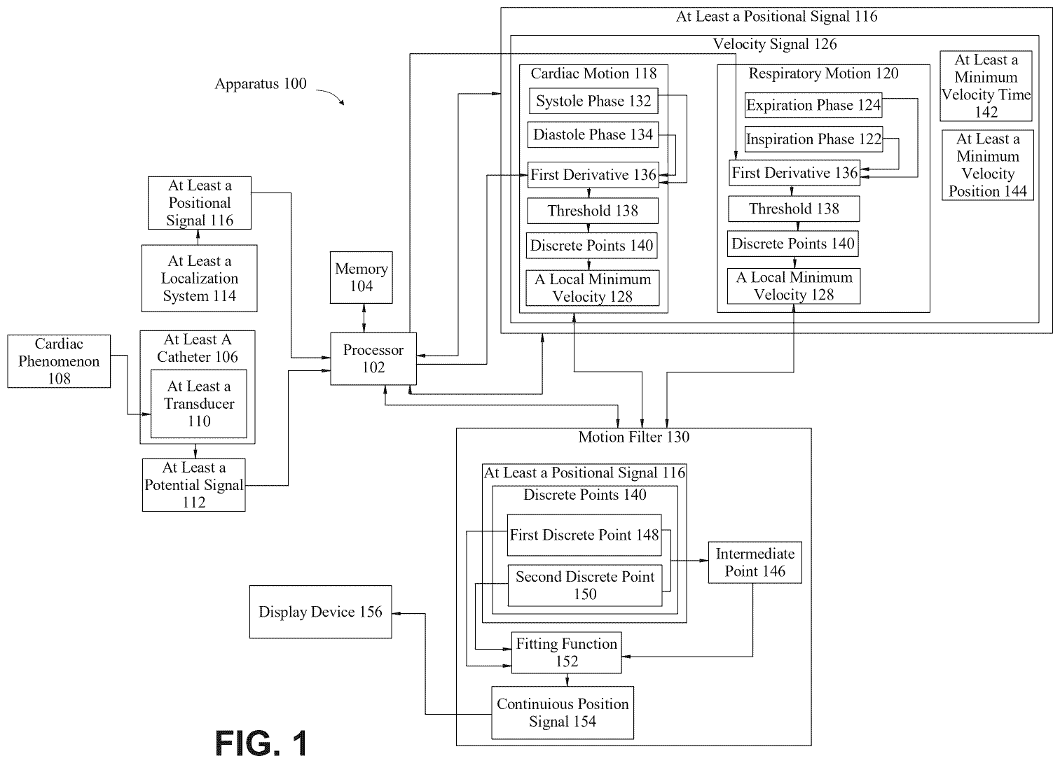

Referring now to FIG. 1, an exemplary embodiment of apparatus 100 for determining a potential signal using catheter velocity during electro-anatomical mapping is illustrated. Apparatus 100 may include a processor 102 communicatively connected to a memory 104. As used in this disclosure, “communicatively connected” means connected by way of a connection, attachment, or linkage between two or more relata which allows for reception and/or transmittance of information therebetween. For example, and without limitation, this connection may be wired or wireless, direct or indirect, and between two or more components, circuits, devices, systems, and the like, which allows for reception and/or transmittance of data and/or signal(s) therebetween. Data and/or signals there between may include, without limitation, electrical, electromagnetic, magnetic, video, audio, radio and microwave data and/or signals, combinations thereof, and the like, among others. A communicative connection may be achieved, for example and without limitation, through wired or wireless electronic, digital or analog, communication, either directly or by way of one or more intervening devices or components. Further, communicative connection may include electrically coupling or connecting at least an output of one device, component, or circuit to at least an input of another device, component, or circuit. For example, and without limitation, via a bus or other facility for intercommunication between elements of a computing device. Communicative connecting May also include indirect connections via, for example and without limitation, wireless connection, radio communication, low power wide area network, optical communication, magnetic, capacitive, or optical coupling, and the like. In some instances, the terminology “communicatively coupled” may be used in place of communicatively connected in this disclosure.

With continued reference to FIG. 1, memory 104 may include a primary memory and a secondary memory. “Primary memory” also known as “random access memory” (RAM) for the purposes of this disclosure is a short-term storage device in which information is processed. In one or more embodiments, during use of the computing device, instructions and/or information may be transmitted to primary memory wherein information may be processed. In one or more embodiments, information may only be populated within primary memory while a particular software is running. In one or more embodiments, information within primary memory is wiped and/or removed after the computing device has been turned off and/or use of a software has been terminated. In one or more embodiments, primary memory may be referred to as “Volatile memory” wherein the volatile memory only holds information while data is being used and/or processed. In one or more embodiments, volatile memory may lose information after a loss of power. “Secondary memory” also known as “storage,” “hard disk drive” and the like for the purposes of this disclosure is a long-term storage device in which an operating system and other information is stored. In one or remote embodiments, information may be retrieved from secondary memory and transmitted to primary memory during use. In one or more embodiments, secondary memory may be referred to as non-volatile memory wherein information is preserved even during a loss of power. In one or more embodiments, data within secondary memory cannot be accessed by processor. In one or more embodiments, data is transferred from secondary to primary memory wherein processor 102 may access the information from primary memory.

Still referring to FIG. 1, apparatus 100 may include a database. The database may include a remote database. The database may be implemented, without limitation, as a relational database, a key-value retrieval database such as a NOSQL database, or any other format or structure for use as database that a person skilled in the art would recognize as suitable upon review of the entirety of this disclosure. The database may alternatively or additionally be implemented using a distributed data storage protocol and/or data structure, such as a distributed hash table or the like. The database may include a plurality of data entries and/or records as described above. Data entries in database may be flagged with or linked to one or more additional elements of information, which may be reflected in data entry cells and/or in linked tables such as tables related by one or more indices in a relational database. Persons skilled in the art, upon reviewing the entirety of this disclosure, will be aware of various ways in which data entries in database may store, retrieve, organize, and/or reflect data and/or records.

With continued reference to FIG. 1, apparatus 100 may include and/or be communicatively connected to a server, such as but not limited to, a remote server, a cloud server, a network server and the like. In one or more embodiments, the computing device may be configured to transmit one or more processes to be executed by server. In one or more embodiments, server may contain additional and/or increased processor power wherein one or more processes as described below may be performed by server. For example, and without limitation, one or more processes associated with machine learning may be performed by network server, wherein data is transmitted to server, processed and transmitted back to computing device. In one or more embodiments, server may be configured to perform one or more processes as described below to allow for increased computational power and/or decreased power usage by the apparatus computing device. In one or more embodiments, computing device may transmit processes to server wherein computing device may conserve power or energy.

Further referring to FIG. 1, apparatus 100 may include any “computing device” as described in this disclosure, including without limitation a microcontroller, microprocessor, digital signal processor (DSP) and/or system on a chip (SoC) as described in this disclosure. Apparatus 100 may include, be included in, and/or communicate with a mobile device such as a mobile telephone or smartphone. Apparatus 100 may include a single computing device operating independently, or may include two or more computing devices operating in concert, in parallel, sequentially or the like; two or more computing devices may be included together in a single computing device or in two or more computing devices. Apparatus 100 may interface or communicate with one or more additional devices as described below in further detail via a network interface device. Network interface device may be utilized for connecting processor 102 to one or more of a variety of networks, and one or more devices. Examples of a network interface device include, but are not limited to, a network interface card (e.g., a mobile network interface card, a LAN card), a modem, and any combination thereof. Examples of a network include, but are not limited to, a wide area network (e.g., the Internet, an enterprise network), a local area network (e.g., a network associated with an office, a building, a campus or other relatively small geographic space), a telephone network, a data network associated with a telephone/voice provider (e.g., a mobile communications provider data and/or voice network), a direct connection between two computing devices, and any combinations thereof. A network may employ a wired and/or a wireless mode of communication. In general, any network topology may be used. Information (e.g., data, software etc.) may be communicated to and/or from a computer and/or a computing device. Processor 102 may include but is not limited to, for example, a computing device or cluster of computing devices in a first location and a second computing device or cluster of computing devices in a second location. Apparatus 100 may include one or more computing devices dedicated to data storage, security, distribution of traffic for load balancing, and the like. Apparatus 100 may distribute one or more computing tasks as described below across a plurality of computing devices of computing device, which may operate in parallel, in series, redundantly, or in any other manner used for distribution of tasks or memory between computing devices. Apparatus 100 may be implemented, as a non-limiting example, using a “shared nothing” architecture.

With continued reference to FIG. 1, processor 102 may be designed and/or configured to perform any method, method step, or sequence of method steps in any embodiment described in this disclosure, in any order and with any degree of repetition. For instance, processor 102 may be configured to perform a single step or sequence repeatedly until a desired or commanded outcome is achieved; repetition of a step or a sequence of steps may be performed iteratively and/or recursively using outputs of previous repetitions as inputs to subsequent repetitions, aggregating inputs and/or outputs of repetitions to produce an aggregate result, reduction or decrement of one or more variables such as global variables, and/or division of a larger processing task into a set of iteratively addressed smaller processing tasks. Processor 102 may perform any step or sequence of steps as described in this disclosure in parallel, such as simultaneously and/or substantially simultaneously performing a step two or more times using two or more parallel threads, processor cores, or the like; division of tasks between parallel threads and/or processes may be performed according to any protocol suitable for division of tasks between iterations. Persons skilled in the art, upon reviewing the entirety of this disclosure, will be aware of various ways in which steps, sequences of steps, processing tasks, and/or data may be subdivided, shared, or otherwise dealt with using iteration, recursion, and/or parallel processing.

Still referring to FIG. 1, apparatus includes at least a catheter 106 configured for intracardiac use. As used in this disclosure, a “catheter” is a flexible tube inserted into the body to perform various medical procedures. In a non-limiting example, at least a catheter 106 may be used to facilitate the detection and mapping of cardiac activity, providing essential data for apparatus 100 to process and analyze. In a non-limiting example, at least a catheter 106 may be used in procedures such as cardiac ablation or electrophysiological studies to gather detailed information about heart rhythms.

With continued reference to FIG. 1, at least a catheter 106 may include at least a transducer 110 configured to detect cardiac phenomenon 108 and output at least a potential signal, as a function of a cardiac phenomenon. As used in this disclosure, a “cardiac phenomenon” is any physiological or pathological event, activity, or condition related to the function or behavior of the heart that can be detected and measured. This includes but is not limited to electrical signals, mechanical movements, pressure changes, and biochemical processes occurring within the heart or its surrounding tissues. Cardiac phenomena are crucial indicators of heart health and function, and they provide valuable data for diagnosing, monitoring, and treating various cardiac conditions. In a non-limiting example, cardiac phenomenon 108 may refer to the electrical activity associated with the heart's rhythm, such as the depolarization and repolarization of cardiac cells that create the P wave, QRS complex, and T wave observed in an electrocardiogram (ECG). Transducer 110 in at least a catheter 106 may detect these electrical signals and output potential signals corresponding to these cardiac events, enabling real-time monitoring of heart rhythms and the identification of arrhythmias or other electrical abnormalities. In another non-limiting example, cardiac phenomenon 108 may include the mechanical contraction and relaxation of the heart muscle, such as the pressure changes during the systolic and diastolic phases of the cardiac cycle. Transducer 110 may detect variations in intracardiac pressure and generate signals that reflect these pressure changes. This data may be used to assess cardiac output, diagnose conditions like heart failure or valvular heart disease, and guide therapeutic interventions by providing detailed insights into the heart's mechanical function. As used in this disclosure, a “transducer” is a device designed to convert one form of energy into another. In a non-limiting example, transducer 110 may facilitate the measurement, monitoring, and control of various physical quantities. Without limitation, this energy conversion capability may enable transducers to be used for various applications. For instance, without limitation, transducers may be used in ultrasound equipment to transform electrical energy into sound waves and vice versa, creating images of internal body structures. When a transducer converts a quantity of energy to an electrical voltage or an electrical current it is called a sensor. A measurable quantity of energy may include sound pressure, optical intensity, magnetic field intensity, thermal pressure, etc. When a transducer converts an electrical signal into another form of energy such as sound, light, mechanical movement, it is called an actuator. It should be noted that sound is incidentally a pressure field. Actuators allow the use of feedback at the source of the measurements. In a non-limiting embodiment, transducer 110 may detect at least a cardiac phenomenon 108 and output potential signal. In another non-limiting example, transducer 110 may include a plurality of clinical transducers. As used in this disclosure, a “plurality of clinical transducers” is a transducer device used in the medical field to measure, analyze, and/or quantify electrical signals in a body. As used in this disclosure, a “potential signal” is the electrical signal generated and output by a transducer in response to detecting cardiac phenomenon 108 within the heart. Potential signal 112 may be indicative of the heart's electrical activity, which may be used for diagnostic or monitoring purposes. Potential signal 112 may represent variations in electrical potential that occur as the heart undergoes its rhythmic contractions and relaxations, providing valuable data on the cardiac cycle and function. In a non-limiting example, potential signal 112 may be generated by transducer 110 embedded in at least a catheter 106 during an electrophysiological study. When at least a catheter 106 is positioned intracardially, transducer 110 detects electrical impulses corresponding to the depolarization and repolarization phases of the cardiac cycle. Potential signal 112 may then be transmitted to an external monitoring system where it is displayed as an electrocardiogram (ECG) tracing. Continuing, the tracing may allow cardiologists to analyze the electrical activity and identify abnormalities such as arrhythmias or conduction blockages. In another non-limiting example, potential signal 112 may be used in a real-time cardiac monitoring system during a surgical procedure. Continuing, as at least a catheter 106 transducer 110 detects changes in intracardiac electrical activity, potential signal 112 is continuously sent to a monitoring device. Without limitation, surgeons may use this real-time data to make informed decisions about interventions, ensuring that the heart remains stable and functions properly throughout given procedure.

Still referring to FIG. 1, apparatus includes at least a localization system 114 configured to detect at least a position signal as a function of a location of the at least a catheter. As used in this disclosure, a “localization system” is a specialized apparatus designed to detect and determine the position of a device within a body or environment. In a non-limiting example, at least a localization system 114 may enhance the safety and efficacy of catheter-based interventions by providing critical spatial information. As used in this disclosure, a “position signal” is a signal generated by localization system 114 to determine the location of catheter 106 within the body.

With continued reference to FIG. 1, the at least a localization system 114 may include one or more of an electromagnetic localization system, an ultrasound-based localization system, an optical localization system, and an impedance-based localization system. As used in this disclosure, an “electromagnetic localization system” is a type of localization technology that uses electromagnetic fields to determine the precise position and orientation of objects within a given space. This system typically involves generating a low-frequency electromagnetic field in the area of interest, and then tracking the position of sensors or coils that respond to this field. The sensors are often integrated into catheters or other medical instruments, allowing for accurate real-time tracking of their location and movement within the body. In the context of electroanatomic mapping, the electromagnetic field-based system enables the precise localization of at least a catheter 106 tip within the heart. This may be achieved by placing electromagnetic field generators around the patient and using sensors on at least a catheter 106 to detect the field. The system may calculate the exact position and orientation of at least a catheter 106 by measuring the electromagnetic field's strength and direction at the sensor's location. This information may be transmitted to processor 102, which may use it to construct a detailed, three-dimensional map of the heart's anatomy. This technology may provide essential information for guiding medical procedures such as catheter ablation, where precise navigation within the heart is critical. By providing accurate and real-time positional data, the electromagnetic field-based system may ensure that at least a catheter 106 can be maneuvered safely and effectively to target areas of abnormal electrical activity, thereby improving the outcomes of the procedure.

With continued reference to FIG. 1, as used in this disclosure, an “ultrasound-based localization system” is a method used to determine the position and movement of objects within the body by employing high-frequency sound waves. The ultrasound-based localization system may involve the use of an ultrasound transducer that emits sound waves, which then reflect off internal structures and are captured by transducer 110 or other sensors. Continuing, the reflected sound waves are processed to create real-time images or data points that represent the location and motion of the tracked object, such as catheter 106 or other medical instruments. ultrasound-based localization system may be particularly useful in medical procedures because it provides real-time, non-invasive visualization of internal body structures. In a non-limiting example, an ultrasound-based localization system may operate by emitting high-frequency sound waves from a transducer 110 towards a target object, such as catheter 106 tip. Continuing, these sound waves travel through the medium (e.g., human tissue) and reflect back to transducer 110 upon hitting the object. Continuing, the system may then calculate the distance to the target based on the time it takes for the echoes to return. Continuing, using multiple transducers placed at known positions, the ultrasound-based localization system may triangulate the exact location of the target object in three-dimensional space. Continuing, the ultrasound waves' time-of-flight data is processed through sophisticated algorithms that account for the speed of sound in the medium, ensuring accurate and real-time localization of the target. In another non-limiting example, the ultrasound-based localization system may integrate Doppler shift measurements to enhance accuracy in dynamic environments. When the target object is moving, the frequency of the reflected ultrasound waves changes proportionally to the object's velocity. The ultrasound-based localization system may capture these frequency shifts and compute the object's speed and direction of movement. By combining the positional data from time-of-flight measurements with velocity data from Doppler shifts, the ultrasound-based localization system may provide a comprehensive tracking solution.

With continued reference to FIG. 1, the ultrasound-based localization system may allow clinicians to guide instruments accurately within the body, enhancing the precision and safety of procedures like catheter ablation, biopsies, or other interventions. The ultrasound-based localization system may be integrated with other systems to provide comprehensive spatial and functional mapping of the area being treated. For example, at least a localization system 114 may utilize ultrasound technology, where an array of ultrasound transducers is positioned around the patient. At least a catheter 106, may be fitted with miniature ultrasound receivers, detects the emitted ultrasound waves. At least a localization system 114 may calculate at least a catheter 106 position based on the time it takes for the ultrasound waves to reach the receivers, allowing for precise localization of at least a catheter 106 tip during a procedure. In another non-limiting example, the ultrasound-based localization system may be employed in conjunction with advanced imaging techniques to enhance the accuracy of the localization process. Without limitation, the ultrasound-based localization system may combine ultrasound data with magnetic resonance imaging (MRI) or computed tomography (CT) scans, the system can provide a more detailed and precise map of the internal structures. Continuing, the hybrid approach may allow for better visualization of complex anatomical regions, aiding in the planning and execution of intricate medical procedures. The integration of multiple imaging modalities ensures that localization system 114 can adapt to various clinical scenarios, offering flexibility and improved outcomes for patients.

With continued reference to FIG. 1, in a non-limiting example, the ultrasound-based localization system may be the same or substantially the same as the localization system described in attorney docket number 1518-103USUI, U.S. patent application Ser. No. 18/376,688, filed on Oct. 4, 2023, titled “APPARATUS AND METHODS FOR GENERATING A THREE-DIMENSIONAL (3D) MODEL OF CARDIAC ANATOMY VIA MACHINE-LEARNING,” which is incorporated by reference herein in its entirety.

With continued reference to FIG. 1, in a non-limiting example, the ultrasound-based localization system may be the same or substantially the same as the localization system described in attorney docket number 1518-104USUI, U.S. patent application Ser. No. 18/389,513, filed on Nov. 14, 2023, titled “APPARATUS AND METHODS FOR SYNTHETIZING MEDICAL IMAGES,” which is incorporated by reference herein in its entirety.

With continued reference to FIG. 1, in a non-limiting example, the ultrasound-based localization system may be the same or substantially the same as the localization system described in attorney docket number 1518-105USUI, U.S. patent application Ser. No. 18/426,604, filed on Jan. 30, 2024, titled “APPARATUS AND METHOD FOR GENERATING A THREE-DIMENSIONAL (3D) MODEL OF CARDIAC ANATOMY BASED ON MODEL UNCERTAINTY,” which is incorporated by reference herein in its entirety.

With continued reference to FIG. 1, in a non-limiting example, the ultrasound-based localization system may be the same or substantially the same as the localization system described in attorney docket number 1518-116USUI, U.S. patent application Ser. No. 18/648,138, filed on Apr. 26, 2024, titled “APPARATUS AND METHODS FOR VISUALIZATION WITHIN A THREE-DIMENSIONAL MODEL USING NEURAL NETWORKS,” which is incorporated by reference herein in its entirety.

With continued reference to FIG. 1, as used in this disclosure, “optical localization system” is a method of determining the position and movement of objects using light, typically through the use of cameras and other optical sensors. The optical localization system may capture visual data from the tracked object and processes this information to calculate its precise location and trajectory in real-time. In an optical tracking system, reflective markers or LED lights may be attached to the object being tracked, such as catheter 106 tip. Cameras positioned around the area capture the light reflected or emitted by these markers, and software algorithms analyze the captured images to triangulate the exact position of the markers. This data may be transmitted to processor 102, which integrates it with other signals to create a comprehensive map of the object's movement within the heart. This method is highly accurate and provides detailed spatial information, making it particularly useful in medical applications where precise positioning is crucial. The optical localization system may be used in conjunction with other localization methods to enhance the overall accuracy and reliability of the electroanatomic mapping system.

With continued reference to FIG. 1, as used in this disclosure, “impedance-based localization system” is a technique used to determine the position of catheter 106 or other medical device within the body by measuring the electrical impedance between the device and electrodes placed on the patient's body. The impedance-based localization system may involve passing a small, alternating current through the body and measuring the resulting voltage at different points, allowing the system to calculate the impedance. The impedance-based localization system may then use these impedance measurements to triangulate the exact position of at least a catheter 106 tip within the heart or other body cavities. Impedance varies with the distance and the type of tissue between at least a catheter 106 and the electrodes, enabling precise tracking of the device's location. This technique may be particularly useful in electroanatomic mapping and other procedures requiring accurate real-time positioning of medical instruments within the body. In a non-limiting example, the impedance-based localization system may inject a low-frequency electrical current through electrodes placed on the body of a patient. Continuing, the current may flow through the body tissues, which have varying impedance characteristics. Continuing, by measuring the voltage differences at various points, the impedance-based localization system can determine the impedance values across different regions. These impedance values are then processed using algorithms to create a detailed map of the internal body structures, allowing for precise localization of medical instruments or implants. In another non-limiting example, the impedance-based localization system may be employed during a minimally invasive surgical procedure. For example, small electrodes may be attached to the tip of catheter 106 or endoscope are introduced into the patient's body. Continuing, as catheter 106 moves through the tissues, the impedance-based localization system continuously measures impedance changes. Continuing, these measurements are transmitted to a computer, which dynamically updates the location of catheter 106 on a real-time 3D model of the patient's anatomy. Continuing, this allows the surgeon to navigate the instrument accurately without the need for extensive imaging equipment, thereby reducing radiation exposure and procedure time. In another non-limiting example, the impedance-based localization system may be used for cardiac electrophysiology studies. For example, the impedance-based localization system may help to map the electrical activity of the heart by placing multiple electrodes on the cardiac tissue. Continuing, as the heart beats, the impedance-based localization system may record the impedance changes corresponding to the electrical impulses. These data are then analyzed to identify abnormal electrical pathways that could be causing arrhythmias. This enables the cardiologist to accurately target and ablate problematic tissue areas, enhancing the precision and effectiveness of the treatment.

With continued reference to FIG. 1, in a non-limiting example, the ultrasound-based localization system may be the same or substantially the same as the localization system described in attorney docket number 1518-160USUI, U.S. patent application Ser. No. 18/764,853, filed on Jul. 5, 2024, titled “SYSTEM AND METHOD FOR LOCATING A MEDICAL DEVICE USING AN ELECTRICAL FIELD CREATION,” which is incorporated by reference herein in its entirety.

With continued reference to FIG. 1, in a non-limiting example, the ultrasound-based localization system may be the same or substantially the same as the localization system described in attorney docket number 1518-158USUI, U.S. patent application Ser. No. 18/784,042, filed on Jul. 25, 2024, titled “APPARATUS AND A METHOD FOR THE GENERATION OF AN IMPEDANCE MODEL OF A BIOLOGICAL CHAMBER” which is incorporated by reference herein in its entirety.

With continued reference to FIG. 1, in a non-limiting example, position signal 116 may be generated using electromagnetic fields, ultrasound, or other tracking technologies to provide real-time spatial information about at least a at least a catheter 106 position. In a non-limiting example, apparatus 100 may employ other tracking technologies, such as optical tracking or impedance-based localization, to generate position signal 116. Optical tracking uses cameras and reflective markers on at least a catheter 106 to capture its movement and position, while impedance-based localization measures electrical impedance differences between at least a catheter 106 and the body tissues. These methods provide accurate real-time spatial information that processor 102 uses alongside potential signal 112.

With continued reference to FIG. 1, processor 102 may be configured to receive at least a potential signal 112 from at least a transducer 110 and at least a position signal 116 from the at least a localization system 114. In a non-limiting example, receiving both potential signal 112 and the position signal, processor 102 may combine these data streams to perform comprehensive analyses of the heart's electrical activity and its spatial context. Processor 102 may combine the time stamps of both data streams. Continuing, by aligning the time points, it may ensure that each electrical signal is paired with the precise location in the heart where it was recorded. Processor 102 may combine these data streams by mapping the electrical activity data points onto their corresponding spatial locations within the heart's anatomy. Continuing, by combining the data streams it may allow for the identification of regions with abnormal electrical activity, potentially guiding therapeutic interventions such as catheter ablation. Processor 102 may also use this combined data to track the catheter's movement and ensure that data is collected during periods of minimal motion, thereby improving the accuracy and reliability of the electro-anatomical maps.

With continued reference to FIG. 1, processor 102 may be configured to discretize at least a potential signal 112 and at least a position signal 116. As used in this disclosure, “discretizing” is the process of converting continuous signals or data into a finite set of discrete points or values. In a non-limiting example, discretizing may involve sampling the continuous signal at specific intervals to create a series of distinct data points that can be more easily processed and analyzed by digital systems. Discretizing may be used in various applications including, without limitation, signal processing, data analysis, and digital communication, as it allows for the representation of continuous phenomena in a form that can be handled by digital computers and other electronic devices. In a non-limiting example, discretizing may involve selecting a sample rate to capture a series of discrete points from the continuous electrical signals detected by a catheter's transducer. Continuing, these discrete points are then quantized, meaning they are assigned specific numerical values that approximate the original continuous signal. Continuing, this process may create a digital representations of the heart's electrical activity, which can be further analyzed to identify patterns, detect abnormalities, and guide therapeutic interventions. In a non-limiting example, discretizing may play a crucial role in the integration of positional data from localization systems. For example, by sampling the continuous position signals at regular intervals, the system can generate a series of discrete positional data points that represent the catheter's movement within the heart. Continuing, these discrete points can then be used to track the catheter's trajectory, correlate its position with the detected electrical signals, and ensure accurate data collection during periods of minimal motion. Continuing, this enhances the overall accuracy and reliability of the electro-anatomical maps, providing valuable insights for medical procedures.

With continued reference to FIG. 1, discretizing may include selecting a sample rate for sampling a plurality of discrete points from at least a potential signal 112 and at least a position signal 116 and quantizing the plurality of discrete points. As used in this disclosure, a “sample rate” is the frequency at which a continuous signal is sampled to convert it into a series of discrete data points. Without limitation, the sample rate may be measured in samples per second (Hz) and determines how often the signal is measured and recorded. In a non-limiting example, a higher sample rate may capture more data points within a given time period, providing a more detailed representation of the original continuous signal. Conversely, a lower sample rate may capture fewer data points, which may result in a less accurate representation. In a non-limiting example, the sample rate may be chosen based on the Nyquist Theorem, which states that the sampling rate must be at least twice the highest frequency present in the signal to accurately capture all the relevant information without aliasing. For instance, if the highest frequency in potential signal 112 is 100 Hz, the sample rate should be at least 200 samples per second. Without limitation, once the sample rate is selected, the system begins sampling the potential and position signals at this rate, resulting in a series of discrete data points that represent the signals at specific intervals. This process involves capturing instantaneous values of the signals at each sample point, effectively converting the continuous-time signals into discrete-time signals. These discrete points are then stored in a digital format for further processing and analysis. The precision of this discretization process directly impacts the accuracy and fidelity of the reconstructed signals and the subsequent analysis performed on them. Quantizing the plurality of discrete points may involve mapping the continuous amplitude values of the sampled points to a finite set of discrete levels. This may be achieved through an analog-to-digital converter (ADC) that converts the sampled analog signals into digital values. The number of quantization levels depends on the bit depth of the ADC; for example, an 8-bit ADC provides 256 distinct levels, while a 16-bit ADC provides 65,536 levels. This quantization process may introduce a small error, known as quantization error, which is the difference between the actual analog value and the nearest quantization level. The resolution of the quantization determines the precision of the digital representation of the signals and is crucial for maintaining the integrity of the original continuous signals in the digital domain.

With continued reference to FIG. 1, at least a position signal 116 may include cardiac motion 118 and respiratory motion 120, wherein cardiac motion 118 comprises systole phase 132 and diastole phase 134 and respiratory motion 120 comprises inspiration phase 122 and expiration phase 124. As used in this disclosure, “cardiac motion” is the physical movement of the heart during its contraction and relaxation cycles. In a non-limiting example, cardiac motion may include the mechanical activities associated with the systolic and diastolic phases of the cardiac cycle, such as the ejection of blood from the ventricles and the filling of the atria. In a non-limiting example, cardiac motion may cause motion artifacts in potential signal 112, which can affect the accuracy of beat detection and mapping. In a non-limiting example, motion filter 130, as discussed in more detail below, may be used to detect and compensate for these motion artifacts, ensuring that potential signal 112 accurately reflects the underlying cardiac activity. As used in this disclosure, “systole phase” is the phase of the cardiac cycle during which the heart muscle contracts to pump blood out of the chambers. Specifically, systole involves the contraction of the ventricles, the lower chambers of the heart, which forces blood into the arteries. The right ventricle pumps deoxygenated blood into the pulmonary artery, leading to the lungs, while the left ventricle pumps oxygenated blood into the aorta, supplying the rest of the body. Systole begins with the depolarization of the ventricles, which is triggered by electrical impulses originating from the sinoatrial (SA) node and conducted through the atrioventricular (AV) node. This electrical activity causes the ventricular muscle fibers to contract, increasing the pressure within the ventricles. When the pressure exceeds that in the adjacent arteries, the semilunar valves (the aortic and pulmonary valves) open, allowing blood to be ejected from the heart. The systolic phase is crucial for maintaining effective circulation and ensuring that oxygen-rich blood reaches the tissues and organs. Without limitation, systole phase 132 is important for accurately interpreting the heart's electrical signals and mechanical movements. The contraction during systole may introduce motion artifacts into the data collected by a catheter's transducer, making it essential to account for this phase when analyzing the heart's electrical activity and creating detailed maps for diagnostic and therapeutic purposes. As used in this disclosure, “diastole phase” is the phase of the cardiac cycle during which the heart muscle relaxes after contraction, allowing the chambers to fill with blood. Diastole involves the relaxation of the ventricles, the lower chambers of the heart, which decreases the pressure within these chambers and permits blood to flow in from the atria, the upper chambers of the heart. This phase is essential for ensuring that the heart is adequately filled with blood before the next contraction. Diastole begins with the repolarization of the ventricles, which follows the contraction phase (systole). As the ventricular muscle fibers relax, the pressure within the ventricles drops below that of the atria, causing the atrioventricular (AV) valves (the mitral and tricuspid valves) to open. This allows blood to flow passively from the atria into the ventricles. The diastolic phase is further divided into early diastole, when the ventricles are rapidly filling, and late diastole, which includes atrial contraction to push additional blood into the ventricles. The diastolic phase is crucial for maintaining effective cardiac function and ensuring that the heart is properly filled with blood for the next contraction. Without limitation, diastole phase 134 is important for accurately interpreting the heart's electrical signals and mechanical movements. The relaxation during diastole may provide periods of minimal motion, which are ideal for collecting accurate and reliable data. By focusing on these periods, apparatus 100 may reduce motion artifacts and improve the precision of the electro-anatomical maps used for diagnosing and treating various cardiac conditions.

With continued reference to FIG. 1, as used in this disclosure, “respiratory motion” is the physical movement of the chest and abdomen during the process of breathing, which can cause displacement of internal organs, including the heart. In a non-limiting example, respiratory motion 120 may include the expansion and contraction of the lungs during inhalation and exhalation, which can lead to shifts in the position of the heart and other thoracic structures. In a non-limiting example, respiratory motion 120 may cause motion artifacts in potential signal 112, affecting the accuracy of beat detection and mapping. In a non-limiting example, motion filter 130 may be used to detect and compensate for these motion artifacts, ensuring that potential signal 112 accurately reflects the underlying cardiac activity. In a non-limiting example, cardiac motion and respiratory motion 120 may include baseline wander, which can obscure the true cardiac signals and lead to inaccurate mapping. In a non-limiting example, baseline wander may result from patient movement or respiration, causing slow drifts in the signal baseline. As used in this disclosure, “inspiration phase” is the phase of the respiratory cycle during which air is drawn into the lungs. This phase involves the contraction of the diaphragm and intercostal muscles, which increases the volume of the thoracic cavity and decreases the pressure within the lungs relative to the external environment. As a result, air flows into the lungs to equalize the pressure difference. During inspiration phase 122, the diaphragm moves downward, and the rib cage expands outward, creating more space in the thoracic cavity. This expansion allows the lungs to inflate as air is drawn in through the airways. Inspiration phase 122 is essential for oxygenating the blood, as it brings fresh air containing oxygen into the alveoli, where gas exchange occurs. In a non-limiting example, the movement of the diaphragm and the expansion of the thoracic cavity may cause displacement of internal organs, including the heart. Continuing, this respiratory motion 120 may introduce artifacts into the electrical signals detected by a catheter's transducer, affecting the accuracy of the data. Continuing, by accounting for inspiration phase 122, apparatus 100 may better compensate for these motion artifacts, ensuring more accurate and reliable electro-anatomical maps for diagnosing and treating cardiac conditions. As used in this disclosure, “expiration phase” is the phase of the respiratory cycle during which air is expelled from the lungs. This expiration phase involves the relaxation of the diaphragm and intercostal muscles, which decreases the volume of the thoracic cavity and increases the pressure within the lungs relative to the external environment. As a result, air flows out of the lungs to equalize the pressure difference. During expiration phase 124, the diaphragm moves upward, and the rib cage contracts inward, reducing the space in the thoracic cavity. This reduction in volume forces air out of the lungs through the airways. Expiration phase 124 is essential for removing carbon dioxide, a waste product of cellular metabolism, from the body and maintaining proper gas exchange in the lungs. In a non-limiting example, the movement of the diaphragm and the contraction of the thoracic cavity may cause displacement of internal organs, including the heart. Continuing, this respiratory motion 120 may introduce artifacts into the electrical signals detected by a catheter's transducer, affecting the accuracy of the data. Continuing, by accounting for expiration phase 124, apparatus 100 may better compensate for these motion artifacts, ensuring more accurate and reliable electro-anatomical maps for diagnosing and treating cardiac conditions.

Still referring to FIG. 1, processor 102 is configured to derive at least a velocity signal from at least a position signal 116. In a non-limiting example, this may involve applying mathematical, statistical, or computational techniques to transform raw data into meaningful insights or parameters that can be used for further analysis or decision-making. Deriving may be used in signal processing, data analysis, machine learning, and the like. In a non-limiting example, deriving process may involve processing the raw electrical signals detected by a catheter's transducer to obtain specific parameters such as the heart's electrical activity, the position of the catheter, or the velocity of the catheter's movement. For example, deriving a velocity signal from a position signal may involve calculating the rate of change of the position data over time. Continuing, this may be achieved by taking first derivative 136 of the position signal, which provides information about the speed and direction of the catheter's movement.

Still referring to FIG. 1, processor 102 is configured to identify, using at least a velocity signal 126, local minimum velocity 128 and corresponding at least a minimum velocity time 142 and at least a minimum velocity position 144. As used in this disclosure, a “velocity signal” is a signal that represents the speed and direction of an object's movement. Velocity signal 126 may be derived from positional data, where the rate of change of the object's position is calculated to determine its velocity. Velocity signal 126 may provide valuable information about how fast and in which direction the object is moving at any given moment. In various applications, including medical procedures, robotics, and navigation systems, velocity signal 126 may be crucial for tracking and analyzing dynamic movements. In a non-limiting example, velocity signal 126 may be generated by tracking the movement of catheter 106 within the heart. Continuing, localization system 114 may provide continuous positional data of catheter 106, and processor 102 calculates the velocity by determining the rate of change of this positional data over time. Continuing, the resulting velocity signal may indicate how quickly catheter 106 is moving and in which direction, allowing for precise monitoring of its motion within the cardiac environment. Without limitation, this information may be essential for synchronizing data collection with periods of minimal motion, thereby reducing motion artifacts and improving the accuracy of the electro-anatomical maps. Velocity signal 126 may be represented in various forms, such as a time-series graph showing the speed of catheter 106 along different axes (X, Y, and Z) over time. This graphical representation may help in visualizing the movement patterns of catheter 106 and identifying periods of stable or minimal motion. Without limitation, by analyzing velocity signal 126, clinicians and researchers can gain insights into the dynamic behavior of catheter 106, optimize data acquisition processes, and enhance the overall quality of the diagnostic and therapeutic procedures and the like.

With continued reference to FIG. 1, as used in this disclosure, a “local minimum velocity” is a point in a velocity signal where the speed of an object's movement reaches a temporary low value relative to the surrounding points. In a non-limiting example, the local minimum velocity point may not necessarily mean it is the lowest velocity overall, but rather the lowest within a specific segment of the signal. Identifying local minimum velocities may be important for various applications, including, without limitation, medical procedures, robotics, navigation systems, and the like, as these points often represent moments of minimal motion, which can be critical for accurate data collection and analysis. In a non-limiting example, local minimum velocity 128 may be identified by analyzing velocity signal 126 generated from the movement of catheter 106 within the heart. Continuing, processor 102 may calculate the rate of change of catheter 106 position over time to generate velocity signal 126. Continuing, by examining this signal, processor 102 may detect points where the velocity decreases to a local minimum, indicating that catheter 106 is moving very slowly or is nearly stationary. Continuing, these local minimum velocity points may be ideal for collecting electrical signals, as they reduce the likelihood of motion artifacts and improve the accuracy of the electro-anatomical maps. In a non-limiting example processor 102 may use mathematical techniques to calculate the local minimum velocity 128, such as calculating first derivative 136 of velocity signal 126. First derivative may be with respect to time. Continuing, points where first derivative 136 approaches zero may indicate potential local minimum velocities. Additionally, processor 102 may apply threshold-based filtering to refine the identification process, ensuring that only significant reductions in velocity are considered. Continuing, by recording the time and position associated with each local minimum velocity 128, apparatus 100 may synchronize data collection with periods of minimal motion, enhancing the reliability and precision of the diagnostic and therapeutic procedures and the like. Without limitation the synchronization process may involve identifying and recording the times and positions of minimal motion, and then aligning data collection to these periods.

With continued reference to FIG. 1, as used in this disclosure, “minimum velocity time” is a moment in time at which a local minimum velocity occurs in a velocity signal. In a non-limiting example, the time point is identified by analyzing velocity signal 126 to determine when the speed of an object's movement reaches a temporary low value relative to its surrounding points. Identifying the minimum velocity time is crucial for various applications, including medical procedures, robotics, and navigation systems, as it helps pinpoint moments of minimal motion, which are ideal for accurate data collection and analysis. In a non-limiting example, the minimum velocity time may be determined by examining velocity signal 126 generated from the movement of catheter 106 within the heart.

With continued reference to FIG. 1, as used in this disclosure, “at least a minimum velocity position” is a location in space where a local minimum velocity occurs in a velocity signal. This position is identified by analyzing velocity signal 126 to determine where the speed of an object's movement reaches a temporary low value relative to its surrounding points. In a non-limiting example, the minimum velocity position may be determined by examining velocity signal 126 generated from the movement of catheter 106 within the heart. Continuing, localization system 114 may provide continuous positional data of catheter 106, and processor 102 calculates the velocity by determining the rate of change of this positional data over time. Continuing, by analyzing this signal, processor 102 may detect the exact spatial coordinates where the velocity decreases to a local minimum, indicating that catheter 106 is moving very slowly or is nearly stationary. In a non-limiting example, to identify the minimum velocity position, processor 102 may use mathematical techniques such as calculating first derivative 136 of velocity signal 126. For example, points where first derivative 136 approaches zero indicate potential minimum velocity positions. Additionally, processor 102 may apply threshold-based filtering to refine the identification process, ensuring that only significant reductions in velocity are considered. Continuing, by recording the minimum velocity position associated with each local minimum velocity 128, the system can enhance the reliability and precision of the diagnostic and therapeutic procedures and the like. Continuing, processor may calculate the rate of change of catheter 106 position over time to generate velocity signal 126.

With continued reference to FIG. 1, processor 102 is configured to identify a local minimum velocity 128 of catheter 106 for cardiac motion 118, wherein identifying the local minimum velocity 128 may include calculating first derivative 136 of at least a velocity signal 126, determining threshold 138 to filter discrete points 140, and comparing plurality of discrete points 140. Without limitation, first derivative 136 of velocity signal 126 of catheter 106 is calculated. Continuing, by taking first derivative 136, processor 102 transforms the velocity data into a rate of change of velocity, highlighting moments where the speed of catheter 106 either increases or decreases. Continuing, this transformation may allow the identification of points where the velocity transitions from decreasing to increasing, which corresponds to local minimum points in velocity signal 126. Without limitation, following the calculation of first derivative 136, processor 102 may establish threshold 138 to filter out discrete points 140. Continuing, threshold 138 may help in distinguishing changes in velocity from minor fluctuations that might be due to noise or other irrelevant factors. Without limitation, by setting threshold 138, processor 102 may ensure that only meaningful changes in velocity are considered, enhancing the accuracy of the local minimum identification. Continuing, processor 102 may compare plurality of discrete points 140 against the aforementioned threshold. Without limitation, by analyzing these points, processor 102 may identify points that meet the criteria for local minimum velocity 128, ensuring precise and reliable detection of catheter 106 motion relative to cardiac activity.

Still referring to FIG. 1, apparatus 100 processes, using motion filter 130, at least a position signal 116 as a function of at least a minimum velocity time 142 and at least a minimum velocity position 144. As used in this disclosure, a “motion filter” is a computational tool or algorithm configured to process and refine signals by removing or compensating for the effects of motion. Motion filter 130 may analyze motion data, such as velocity signal 126 or at least a position signal 116 and apply techniques to isolate and mitigate the impact of unwanted movements, such as cardiac movement and respiratory movement, thereby enhancing the quality and reliability of the collected data. In a non-limiting example, motion filter 130 may be used to process the position signal of catheter 106 within the heart. Continuing, the heart's continuous motion, due to both cardiac and respiratory activities, can introduce noise and artifacts into the electrical signals detected by catheter 106 transducer 110. Without limitation, motion filter 130 may analyze velocity signal 126 and position signal by identifying periods of minimal motion, such as during diastole phase 134 of the cardiac cycle or between breaths. Continuing, motion filter 130 may process the signals to identify patterns associated with different types of movement, for example, techniques such as Fourier Transform, Wavelet Transform, or other signal processing methods may be used to decompose the signals into their frequency components. Motion filter 130 may distinguish between different types of movements, such as cardiac motion 118 and respiratory motion 120. Without limitation, cardiac motion 118 and respiratory motion 120 may have distinct frequencies and patterns that can be identified separately from other types of motion. Without limitation, motion filter 130 may apply algorithms to isolate and mitigate the impact of unwanted movements by using techniques such as filtering, smoothing, and thresholding to remove or reduce the effects of these unwanted movements on the data. In a non-limiting example, the algorithms motion filter 130 may apply include, low-pass filtering to remove high-frequency noise and retain important signals, and high-pass filtering to isolate and remove low-frequency components like respiratory motion, Kalman filtering to estimate dynamic system states and reduce noise, Wavelet Transform to decompose signals into different frequency components to identify patterns, adaptive filtering to adjust parameters dynamically based on input characteristics, and Principal Component Analysis (PCA) to reduce dimensionality and isolate significant motion components. Continuing, motion filter 130 may correct the data to enhance its quality and reliability by compensating for the identified movements or removing their influence from the overall data set. Without limitation, by focusing on these periods, motion filter 130 may reduce the impact of motion artifacts, ensuring that the collected electrical signals more accurately reflect the underlying cardiac activity.

With continued reference to FIG. 1, processing, using motion filter 130, may include interpolating at least a position signal 116 by sampling plurality of discrete points 140 from at least a position signal 116, averaging intermediate point 146 from first discrete point 148 and second discrete point 150 of plurality of discrete points 140, and fitting function 152 to first discrete point 148 and second discrete point 150 to generate continuous position signal 154. As used in this disclosure, a “discrete point” is a specific, individual data value obtained by sampling a signal at a particular moment in time or space. Discrete points 140 may be the result of discretizing a continuous signal, which involves capturing the signal's value at regular intervals to create a series of distinct data points. Discrete points 140 may be processed and analyzed by digital systems, allowing for the representation of continuous phenomena in a form that can be handled by computers and other electronic devices. In a non-limiting example, discrete points 140 may be derived from the continuous electrical signals detected by catheter 106 transducer and the positional data from localization system 114. For example, the continuous electrical activity of the heart may be sampled at specific intervals to generate discrete points 140 that represent the heart's electrical signals at those moments. Similarly, the continuous movement of catheter 106 within the heart may be sampled to produce discrete positional data points. In a non-limiting example, motion filter 130 may employ various techniques, such as interpolation, averaging, and fitting function 152, to generate a continuous and smoothed position signal from discrete points 140. For example, motion filter 130 may sample plurality of discrete points 140 from position signal 116, average intermediate point 146 between consecutive data points, and fit a mathematical function to the sampled points to create continuous position signal 154. This processed signal can then be used to correlate the cardiac and respiratory motion 120, allowing for the removal of these motions from the positional data. By doing so, motion filter 130 enhances the precision and reliability of the electro-anatomical maps, providing valuable insights for diagnosing and treating cardiac conditions and the like.

With continued reference to FIG. 1, motion filter 130 may be configured to remove cardiac motion 118 and respiratory motion 120 from at least a position signal 116 which may include correlating cardiac motion 118 and respiratory motion 120 to determine a total motion of the position signal. As used in this disclosure, “total motion” is the combined movement of an object resulting from multiple sources of motion, such as cardiac motion, respiratory motion, and any other relevant movements. Total motion encompasses all the dynamic activities that affect the position and orientation of the object over time. Understanding and quantifying total motion is crucial for applications that require precise tracking and analysis, such as medical procedures, robotics, and navigation systems. Without limitation, by analyzing the combined effects of both cardiac and respiratory movements, processor 102 may identify and isolate periods of minimal total motion. Continuing, this allows for the removal of these motion components from the position signal, resulting in a more accurate and stable representation of catheter 106 position within the heart, thereby enhancing the reliability of the electro-anatomical maps.

With continued reference to FIG. 1, processor 102 may use display device 156 to display continuous position signal 154. As used in this disclosure, a “display device” refers to an electronic device that visually presents information to the entity. In some cases, display device may be configured to project or show visual content generated by computers, video devices, or other electronic mechanisms. In some cases, display device may include a liquid crystal display (LCD), a cathode ray tube (CRT), a plasma display, a light emitting diode (LED) display, and any combinations thereof. In a non-limiting example, one or more display devices may vary in size, resolution, technology, and functionality. Display device 156 may be able to show any data elements and/or visual elements as listed above in various formats such as, textural, graphical, video among others, in either monochrome or color. Display device 156 may include, but is not limited to, a smartphone, tablet, laptop, monitor, tablet, and the like. Display device 156 may include a separate device that includes a transparent screen configured to display computer generated images and/or information. In some cases, display device 156 may be configured to present a graphical user interface (GUI) to a user, wherein a user may interact with a GUI. In some cases, a user may view a GUI through display. Additionally, or alternatively, processor 102 be connected to display device 156. In one or more embodiments, transmitting continuous position signal 154 may include displaying continuous position signal 154 at display device 156 using a visual interface.

Exemplary embodiments have been disclosed above and illustrated in the accompanying drawings. It will be understood by those skilled in the art that various changes, omissions and additions may be made to that which is specifically disclosed herein without departing from the spirit and scope of the present invention.

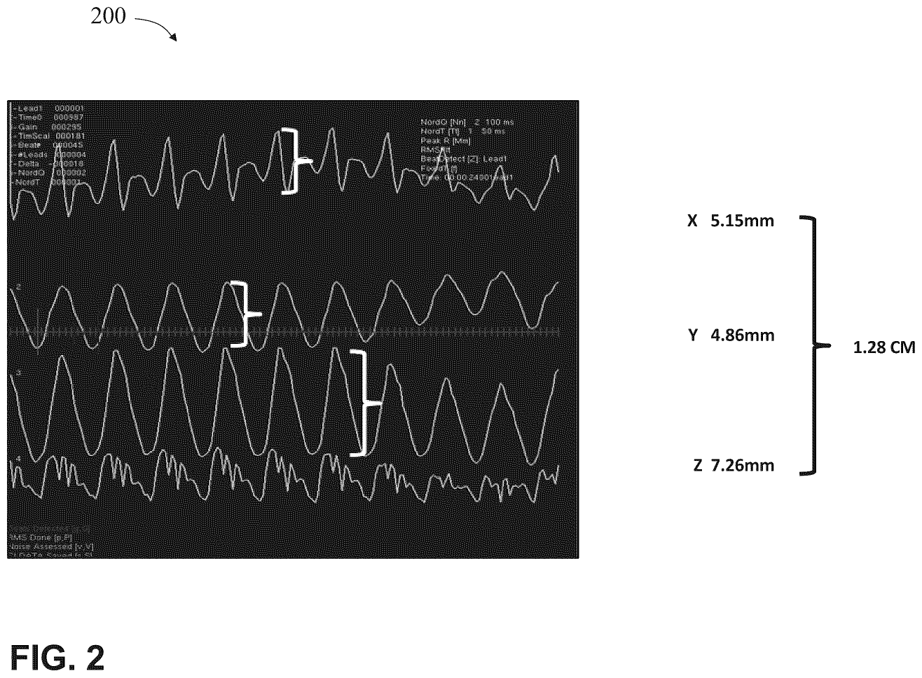

Referring now to FIG. 2, an illustration 200, of a graph of an RA-1 file cardiac motion 4 second window. In an embodiment, the graph includes three distinct waveforms representing cardiac motion data over a 4-second window. In an embodiment, the waveforms are labeled with X, Y, and Z coordinates, indicating the motion in three-dimensional space. In an embodiment, the X coordinate waveform represents the cardiac motion data along the X-axis. In an embodiment, the waveform shows the displacement of the catheter in the X direction over the 4-second window. In an embodiment, the data points are plotted to illustrate the variations in motion due to cardiac activity. In an embodiment, the Y coordinate waveform represents the cardiac motion data along the Y-axis. In an embodiment, the waveform shows the displacement of the catheter in the Y direction over the 4-second window. In an embodiment, the data points are plotted to illustrate the variations in motion due to cardiac activity. In an embodiment, the Z coordinate waveform represents the cardiac motion data along the Z-axis. In an embodiment, the waveform shows the displacement of the catheter in the Z direction over the 4-second window. In an embodiment, the data points are plotted to illustrate the variations in motion due to cardiac activity. In an embodiment, the graph also includes annotations indicating the scale and measurement units. The X, Y, and Z coordinates are measured in millimeters, with the total displacement indicated as 1.28 cm. In an embodiment, the annotations provide additional context for interpreting the cardiac motion data.

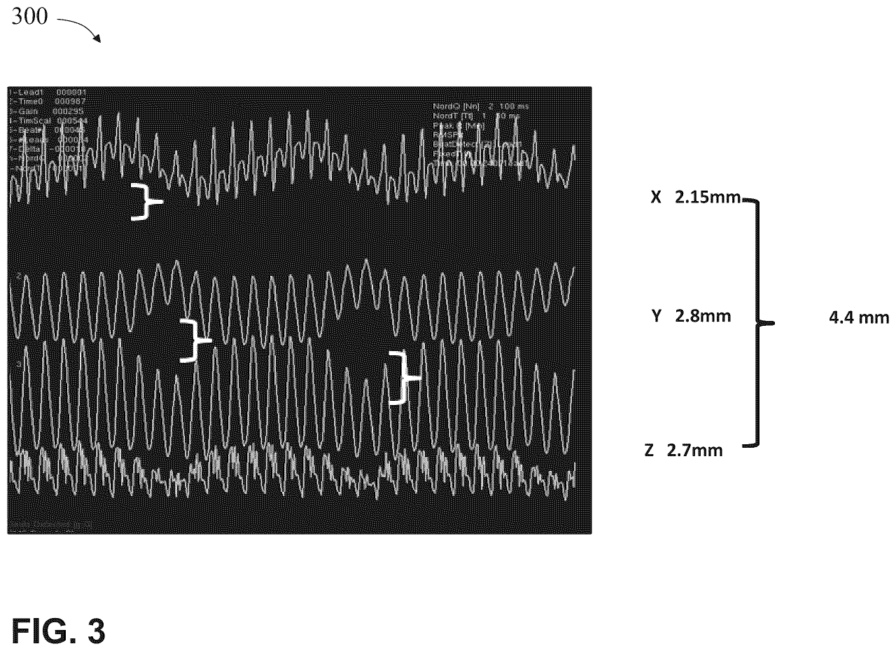

Referring now to FIG. 3, an illustration 300, of a graph of an RA-1 file respiratory motion 13 second window. In an embodiment, the graph includes three distinct waveforms representing respiratory motion data over a 13-second window. In an embodiment, the waveforms are labeled with X, Y, and Z coordinates, indicating the motion in three-dimensional space. In an embodiment, the X coordinate waveform represents the respiratory motion data along the X-axis. In an embodiment, the waveform shows the displacement of the catheter in the X direction over the 13-second window. In an embodiment, the data points are plotted to illustrate the variations in motion due to respiratory activity. In an embodiment, the Y coordinate waveform represents the respiratory motion data along the Y-axis. In an embodiment, the waveform shows the displacement of the catheter in the Y direction over the 13-second window. The data points are plotted to illustrate the variations in motion due to respiratory activity. The Z coordinate waveform represents the respiratory motion data along the Z-axis. In an embodiment, the waveform shows the displacement of the catheter in the Z direction over the 13-second window. In an embodiment, the data points are plotted to illustrate the variations in motion due to respiratory activity. In an embodiment, the graph also includes annotations indicating the scale and measurement units. In an embodiment, the X, Y, and Z coordinates are measured in millimeters, with the total displacement indicated as 4.4 mm. In an embodiment, the annotations provide additional context for interpreting the respiratory motion data.

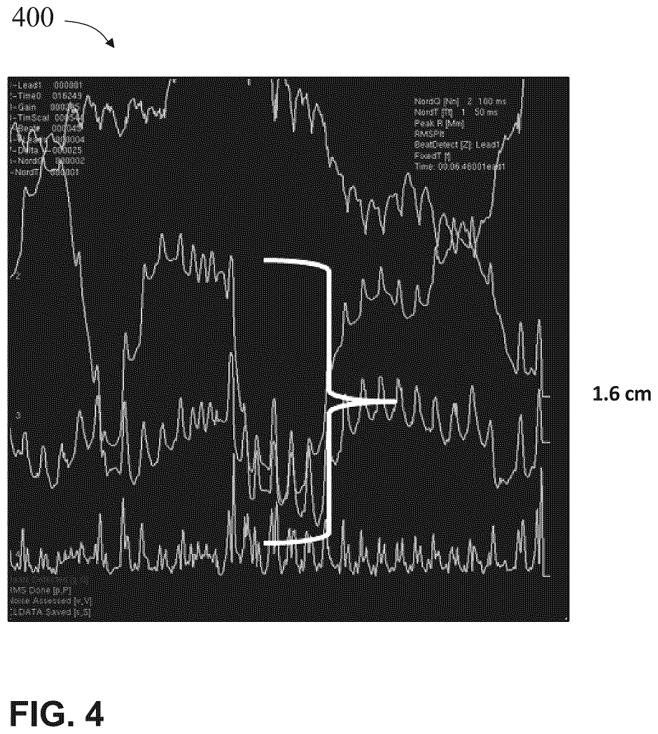

Referring now to FIG. 4, an illustration 400, of a graph of an RA-1 file respiratory motion 13 second window typical catheter location movement. In an embodiment, the graph includes three distinct waveforms representing respiratory motion data over a 13-second window. The waveforms are labeled with X, Y, and Z coordinates, indicating the motion in three-dimensional space. In an embodiment, the X coordinate waveform represents the respiratory motion data along the X-axis. In an embodiment, the waveform shows the displacement of the catheter in the X direction over the 13-second window. The data points are plotted to illustrate the variations in motion due to respiratory activity. In an embodiment, the Y coordinate waveform represents the respiratory motion data along the Y-axis. This waveform shows the displacement of the catheter in the Y direction over the 13-second window. In an embodiment, the data points are plotted to illustrate the variations in motion due to respiratory activity. In an embodiment, the Z coordinate waveform represents the respiratory motion data along the Z-axis. In an embodiment, the waveform shows the displacement of the catheter in the Z direction over the 13-second window. The data points are plotted to illustrate the variations in motion due to respiratory activity. The graph also includes annotations indicating the scale and measurement units. In an embodiment, the X, Y, and Z coordinates are measured in millimeters, with the total displacement indicated as 1.6 cm. The annotations provide additional context for interpreting the respiratory motion data.

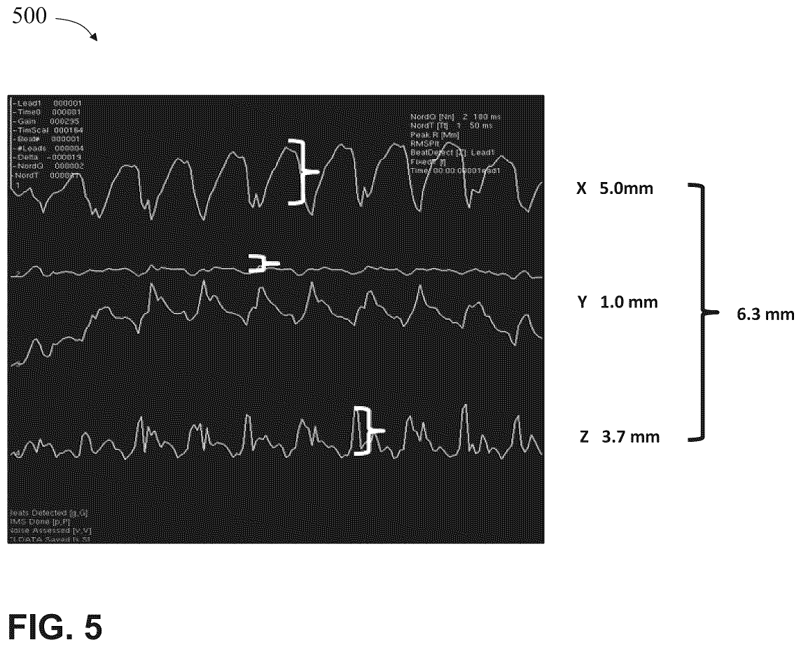

Referring now to FIG. 5, an illustration 500, of a graph of an RV-1 file cardiac motion 4 second window. In an embodiment, the graph includes three distinct waveforms representing cardiac motion data over a 4-second window. In an embodiment, the waveforms are labeled with X, Y, and Z coordinates, indicating the motion in three-dimensional space. In an embodiment, the X coordinate waveform represents the cardiac motion data along the X-axis. In an embodiment, the waveform shows the displacement of the catheter in the X direction over the 4-second window. In an embodiment, the data points are plotted to illustrate the variations in motion due to cardiac activity. In an embodiment, the X coordinate shows a displacement of 5.0 mm. In an embodiment, the Y coordinate waveform represents the cardiac motion data along the Y-axis. In an embodiment, the waveform shows the displacement of the catheter in the Y direction over the 4-second window. In an embodiment, the data points are plotted to illustrate the variations in motion due to cardiac activity. In an embodiment, the Y coordinate shows a displacement of 1.0 mm. In an embodiment, the Z coordinate waveform represents the cardiac motion data along the Z-axis. In an embodiment, the waveform shows the displacement of the catheter in the Z direction over the 4-second window. In an embodiment, the data points are plotted to illustrate the variations in motion due to cardiac activity. The Z coordinate shows a displacement of 3.7 mm. In an embodiment, the graph also includes annotations indicating the scale and measurement units. In an embodiment, the X, Y, and Z coordinates are measured in millimeters, with the total displacement indicated as 6.3 mm. In an embodiment, the annotations provide additional context for interpreting the cardiac motion data.

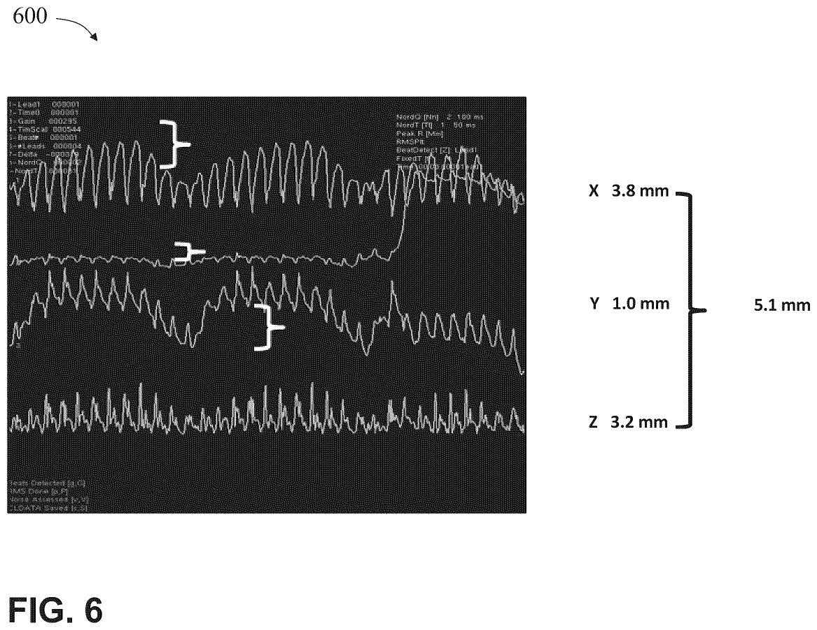

Referring now to FIG. 6, an illustration 600, of a graph of an RV-1 file respiratory motion 13 second window. In an embodiment, the graph includes three distinct waveforms representing respiratory motion data over a 13-second window. In an embodiment, the waveforms are labeled with X, Y, and Z coordinates, indicating the motion in three-dimensional space. In an embodiment, the X coordinate waveform represents the respiratory motion data along the X-axis. This waveform shows the displacement of the catheter in the X direction over the 13-second window. In an embodiment, the data points are plotted to illustrate the variations in motion due to respiratory activity. In an embodiment, the X coordinate shows a displacement of 3.8 mm. In an embodiment, the Y coordinate waveform represents the respiratory motion data along the Y-axis. In an embodiment, the waveform shows the displacement of the catheter in the Y direction over the 13-second window. In an embodiment, the data points are plotted to illustrate the variations in motion due to respiratory activity. In an embodiment, the Y coordinate shows a displacement of 1.0 mm. In an embodiment, the Z coordinate waveform represents the respiratory motion data along the Z-axis. This waveform shows the displacement of the catheter in the Z direction over the 13-second window. The data points are plotted to illustrate the variations in motion due to respiratory activity. The Z coordinate shows a displacement of 3.2 mm. In an embodiment, the graph also includes annotations indicating the scale and measurement units. In an embodiment, the X, Y, and Z coordinates are measured in millimeters, with the total displacement indicated as 5.1 mm. In an embodiment, the annotations provide additional context for interpreting the respiratory motion data.

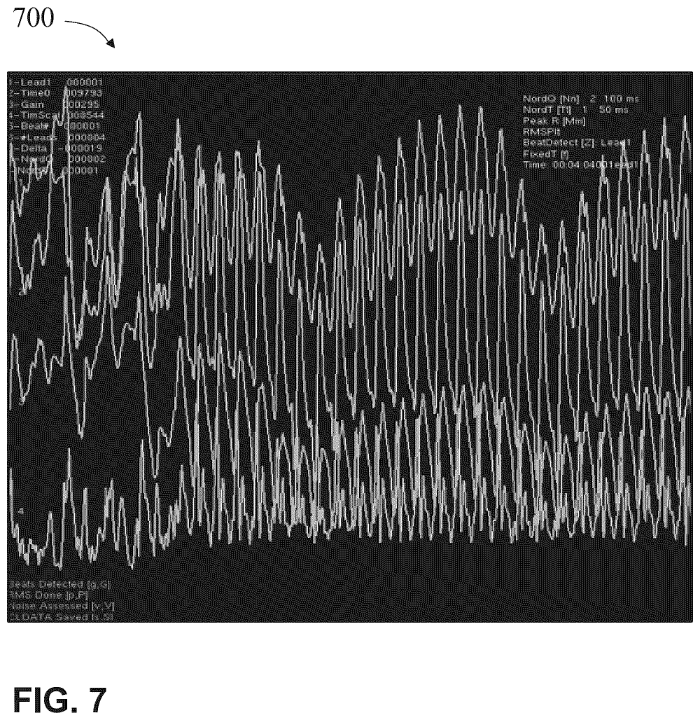

Referring now to FIG. 7 is an illustration 700, of a graph of an RV-1 file all motion 13 second window. In an embodiment, the graph includes three distinct waveforms representing the combined cardiac and respiratory motion data over a 13-second window. In an embodiment, the waveforms are labeled with X, Y, and Z coordinates, indicating the motion in three-dimensional space. In an embodiment, the X coordinate waveform represents the combined motion data along the X-axis. In an embodiment, the waveform shows the displacement of the catheter in the X direction over the 13-second window. In an embodiment, the data points are plotted to illustrate the variations in motion due to both cardiac and respiratory activities. In an embodiment, the Y coordinate waveform represents the combined motion data along the Y-axis.

In an embodiment, the waveform shows the displacement of the catheter in the Y direction over the 13-second window. In an embodiment, the data points are plotted to illustrate the variations in motion due to both cardiac and respiratory activities. In an embodiment, the Z coordinate waveform represents the combined motion data along the Z-axis. In an embodiment, the waveform shows the displacement of the catheter in the Z direction over the 13-second window. In an embodiment, the data points are plotted to illustrate the variations in motion due to both cardiac and respiratory activities. In an embodiment, the graph also includes annotations indicating the scale and measurement units. In an embodiment, the X, Y, and Z coordinates are measured in millimeters. In an embodiment, the annotations provide additional context for interpreting the combined motion data.

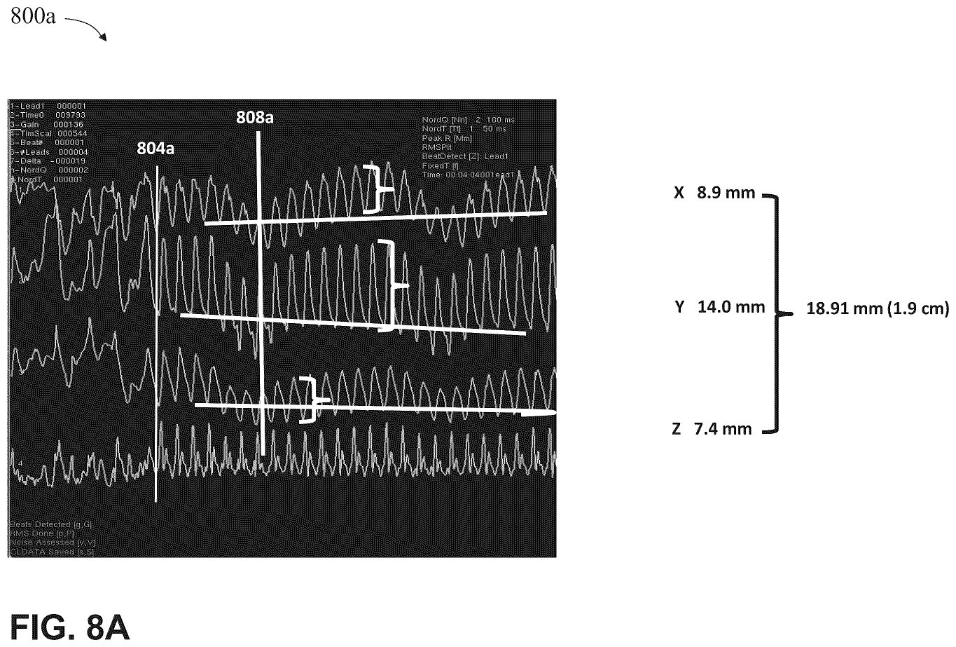

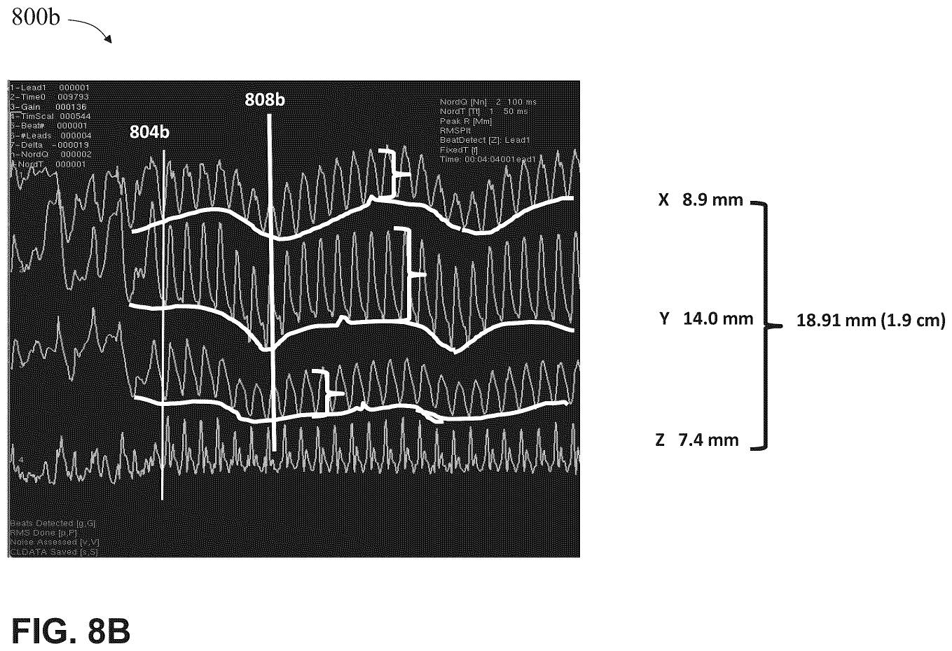

Referring now to FIG. 8A, an illustration 800a, of a graph of an RV-1 file cardiac motion 13 second window showing the smoothed curve. In FIG. 8B, an illustration 800b, of a graph of an RV-1 file cardiac motion 13 second window showing the diastolic envelope. In an embodiment, the graph includes three distinct waveforms representing cardiac motion data over a 13-second window. In an embodiment, the waveforms are labeled with X, Y, and Z coordinates, indicating the motion in three-dimensional space. In an embodiment, the X coordinate waveform represents the cardiac motion data along the X-axis. In an embodiment, the Y coordinate waveform represents the cardiac motion data along the Y-axis. In an embodiment, the Z coordinate waveform represents the cardiac motion data along the Z-axis. In an embodiment, the graph also includes annotations indicating the scale and measurement units, with the X, Y, and Z coordinates measured in millimeters. In an embodiment, the X coordinate waveform shows the displacement of the catheter in the X direction over the 13-second window. In an embodiment, the data points are plotted to illustrate the variations in motion due to cardiac activity. In an embodiment, the X coordinate shows a displacement of 8.9 mm. In an embodiment, the Y coordinate waveform shows the displacement of the catheter in the Y direction over the 13-second window. In an embodiment, the data points are plotted to illustrate the variations in motion due to cardiac activity. In an embodiment, the Y coordinate shows a displacement of 14.0 mm. In an embodiment, the Z coordinate waveform shows the displacement of the catheter in the Z direction over the 13-second window. In an embodiment, the data points are plotted to illustrate the variations in motion due to cardiac activity. In an embodiment, the Z coordinate shows a displacement of 7.4 mm. In an embodiment, the vertical left most line illustrates the end diastole 804a. In an embodiment, the vertical right most line illustrates the peak inspiration 808a.

FIG. 8B shows an illustration 800b of a graph of an RV-1 file cardiac motion 13 second window showing the diastolic envelope. In an embodiment, the graph includes three distinct waveforms representing cardiac motion data over a 13-second window. In an embodiment, the waveforms are labeled with X, Y, and Z coordinates, indicating the motion in three-dimensional space. In an embodiment, the X coordinate waveform represents the cardiac motion data along the X-axis. In an embodiment, the Y coordinate waveform represents the cardiac motion data along the Y-axis. In an embodiment, the Z coordinate waveform represents the cardiac motion data along the Z-axis. In an embodiment, the graph also includes annotations indicating the scale and measurement units, with the X, Y, and Z coordinates measured in millimeters. In an embodiment, the X coordinate waveform shows the displacement of the catheter in the X direction over the 13-second window. In an embodiment, the data points are plotted to illustrate the variations in motion due to cardiac activity. In an embodiment, the X coordinate shows a displacement of 8.9 mm. In an embodiment, the Y coordinate waveform shows the displacement of the catheter in the Y direction over the 13-second window. The data points are plotted to illustrate the variations in motion due to cardiac activity. In an embodiment, the Y coordinate shows a displacement of 14.0 mm. In an embodiment, the Z coordinate waveform shows the displacement of the catheter in the Z direction over the 13-second window. In an embodiment, the data points are plotted to illustrate the variations in motion due to cardiac activity. The Z coordinate shows a displacement of 7.4 mm In an embodiment, the vertical left most line illustrates the end diastole 804b. In an embodiment, the vertical right most line illustrates the peak inspiration 808b.

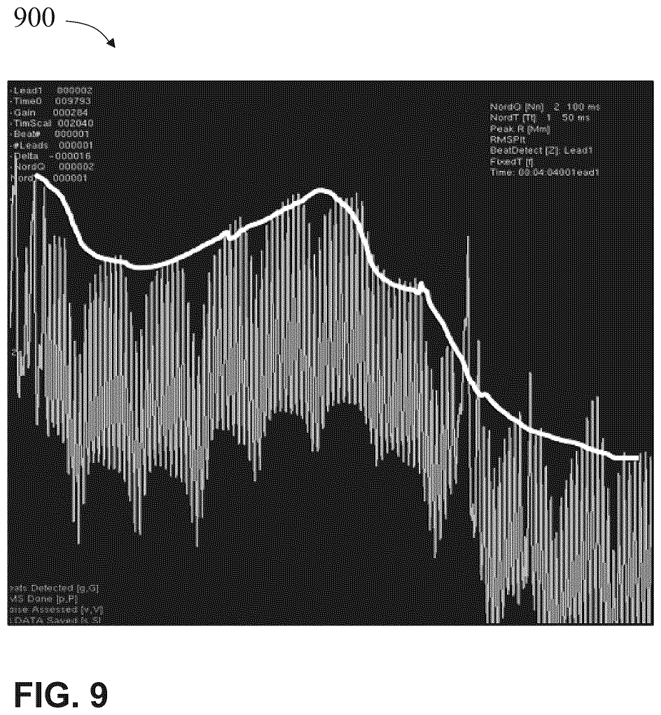

Referring now to FIG. 9, an illustration 900, of a graph of an RV-1 file cardiac motion 95 second window with smoothed Y curve. In an embodiment, the graph shows the cardiac motion data over a 95-second window, with the smoothed Y curve superimposed to highlight the overall trend of the motion data. In an embodiment, the RV-1 file cardiac motion data represents the raw cardiac motion signals collected over the specified time window. In an embodiment, the data is characterized by high-frequency oscillations that correspond to the rapid movements of the heart during cardiac cycles. In an embodiment, the smoothed Y curve is derived from the raw cardiac motion data. This curve is generated by applying a smoothing algorithm to the raw data, which reduces the high-frequency noise and artifacts. In an embodiment, the smoothed Y curve provides a clearer representation of the underlying cardiac motion trend over the 95-second window. In an embodiment, the graph in FIG. 9 may be used to analyze the cardiac motion by comparing the raw data with the smoothed Y curve. In an embodiment, the comparison helps in identifying significant patterns and anomalies in the cardiac motion.

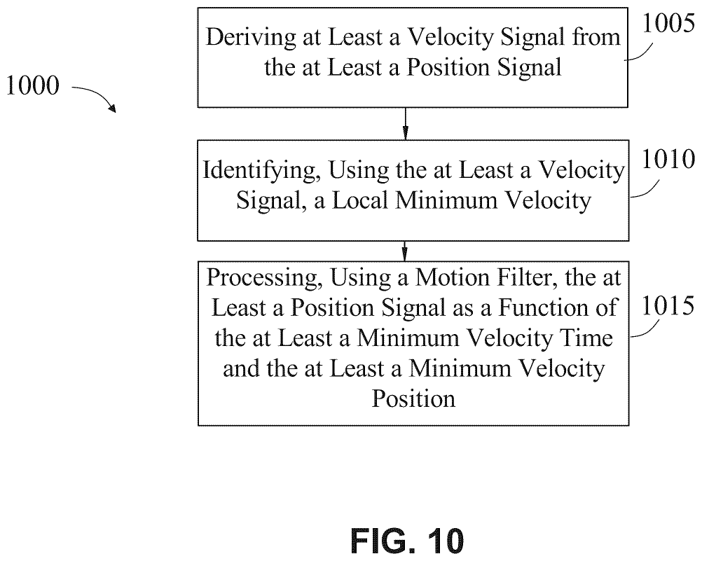

Referring now to FIG. 10, a flow diagram of an exemplary method 1000 for determining a potential signal using catheter velocity during electro-anatomical mapping is illustrated. At step 1005, method 1000 includes deriving, using at least a processor, at least a velocity signal from the at least a position signal. This may be implemented as described and with reference to FIGS. 1-10.

Still referring to FIG. 10, at step 1010, method 1000 includes identifying, using the at least a velocity signal, a local minimum velocity and corresponding at least a minimum velocity time and at least a minimum velocity position. This may be implemented as described and with reference to FIGS. 1-10.

Still referring to FIG. 10, at step 1015, method 1000 includes processing, using a motion filter, the at least a position signal as a function of the at least a minimum velocity time and the at least a minimum velocity position. This may be implemented as described and with reference to FIGS. 1-10.

It is to be noted that any one or more of the aspects and embodiments described herein may be conveniently implemented using one or more machines (e.g., one or more computing devices that are utilized as a user computing device for an electronic document, one or more server devices, such as a document server, etc.) programmed according to the teachings of the present specification, as will be apparent to those of ordinary skill in the computer art. Appropriate software coding can readily be prepared by skilled programmers based on the teachings of the present disclosure, as will be apparent to those of ordinary skill in the software art. Aspects and implementations discussed above employing software and/or software modules may also include appropriate hardware for assisting in the implementation of the machine executable instructions of the software and/or software module.

Such software may be a computer program product that employs a machine-readable storage medium. A machine-readable storage medium may be any medium that is capable of storing and/or encoding a sequence of instructions for execution by a machine (e.g., a computing device) and that causes the machine to perform any one of the methodologies and/or embodiments described herein. Examples of a machine-readable storage medium include, but are not limited to, a magnetic disk, an optical disc (e.g., CD, CD-R, DVD, DVD-R, etc.), a magneto-optical disk, a read-only memory “ROM” device, a random access memory “RAM” device, a magnetic card, an optical card, a solid-state memory device, an EPROM, an EEPROM, and any combinations thereof. A machine-readable medium, as used herein, is intended to include a single medium as well as a collection of physically separate media, such as, for example, a collection of compact discs or one or more hard disk drives in combination with a computer memory. As used herein, a machine-readable storage medium does not include transitory forms of signal transmission.

Such software may also include information (e.g., data) carried as a data signal on a data carrier, such as a carrier wave. For example, machine-executable information may be included as a data-carrying signal embodied in a data carrier in which the signal encodes a sequence of instruction, or portion thereof, for execution by a machine (e.g., a computing device) and any related information (e.g., data structures and data) that causes the machine to perform any one of the methodologies and/or embodiments described herein.

Examples of computing device include, but are not limited to, an electronic book reading device, a computer workstation, a terminal computer, a server computer, a handheld device (e.g., a tablet computer, a smartphone, etc.), a web appliance, a network router, a network switch, a network bridge, any machine capable of executing a sequence of instructions that specify an action to be taken by that machine, and any combinations thereof. In one example, a computing device may include and/or be included in a kiosk.

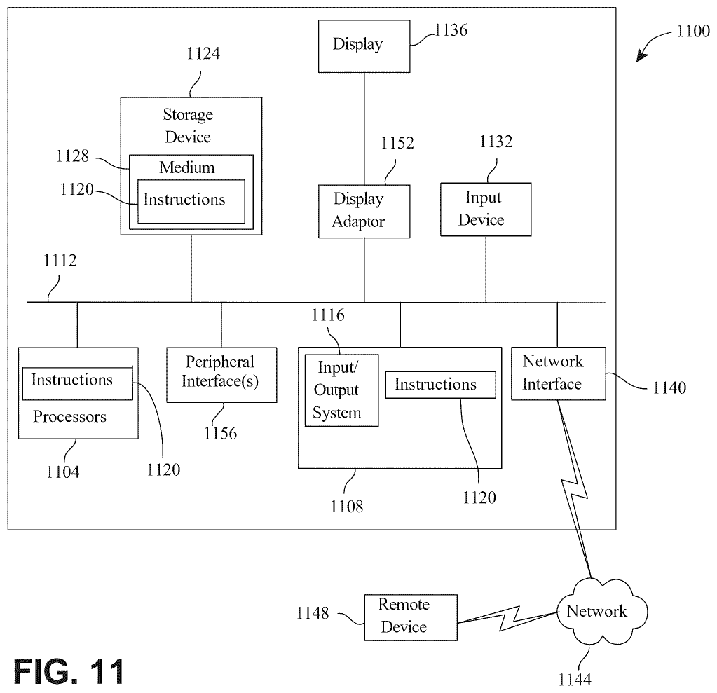

FIG. 11 shows a diagrammatic representation of one embodiment of computing device in the exemplary form of a computer system 1100 within which a set of instructions for causing a control system to perform any one or more of the aspects and/or methodologies of the present disclosure may be executed. It is also contemplated that multiple computing devices may be utilized to implement a specially configured set of instructions for causing one or more of the devices to perform any one or more of the aspects and/or methodologies of the present disclosure. Computer system 1100 includes a processor 1104 and a memory 1108 that communicate with each other, and with other components, via a bus 1112. Bus 1112 may include any of several types of bus structures including, but not limited to, a memory bus, a memory controller, a peripheral bus, a local bus, and any combinations thereof, using any of a variety of bus architectures.

Processor 1104 may include any suitable processor, such as without limitation a processor incorporating logical circuitry for performing arithmetic and logical operations, such as an arithmetic and logic unit (ALU), which may be regulated with a state machine and directed by operational inputs from memory and/or sensors; processor 1104 may be organized according to Von Neumann and/or Harvard architecture as a non-limiting example. Processor 1104 may include, incorporate, and/or be incorporated in, without limitation, a microcontroller, microprocessor, digital signal processor (DSP), Field Programmable Gate Array (FPGA), Complex Programmable Logic Device (CPLD), Graphical Processing Unit (GPU), general purpose GPU, Tensor Processing Unit (TPU), analog or mixed signal processor, Trusted Platform Module (TPM), a floating point unit (FPU), system on module (SOM), and/or system on a chip (SoC).

Memory 1108 may include various components (e.g., machine-readable media) including, but not limited to, a random-access memory component, a read only component, and any combinations thereof. In one example, a basic input/output system 1116 (BIOS), including basic routines that help to transfer information between elements within computer system 1100, such as during start-up, may be stored in memory 1108. Memory 1108 may also include (e.g., stored on one or more machine-readable media) instructions (e.g., software) 1120 embodying any one or more of the aspects and/or methodologies of the present disclosure. In another example, memory 1108 may further include any number of program modules including, but not limited to, an operating system, one or more application programs, other program modules, program data, and any combinations thereof.

Computer system 1100 may also include a storage device 1124. Examples of a storage device (e.g., storage device 1124) include, but are not limited to, a hard disk drive, a magnetic disk drive, an optical disc drive in combination with an optical medium, a solid-state memory device, and any combinations thereof. Storage device 1124 may be connected to bus 1112 by an appropriate interface (not shown). Example interfaces include, but are not limited to, SCSI, advanced technology attachment (ATA), serial ATA, universal serial bus (USB), IEEE 1394 (FIREWIRE), and any combinations thereof. In one example, storage device 1124 (or one or more components thereof) may be removably interfaced with computer system 1100 (e.g., via an external port connector (not shown)). Particularly, storage device 1124 and an associated machine-readable medium 1128 may provide nonvolatile and/or volatile storage of machine-readable instructions, data structures, program modules, and/or other data for computer system 1100. In one example, software 1120 may reside, completely or partially, within machine-readable medium 1128. In another example, software 1120 may reside, completely or partially, within processor 1104.

Computer system 1100 may also include an input device 1132. In one example, a user of computer system 1100 may enter commands and/or other information into computer system 1100 via input device 1132. Examples of an input device 1132 include, but are not limited to, an alpha-numeric input device (e.g., a keyboard), a pointing device, a joystick, a gamepad, an audio input device (e.g., a microphone, a voice response system, etc.), a cursor control device (e.g., a mouse), a touchpad, an optical scanner, a video capture device (e.g., a still camera, a video camera), a touchscreen, and any combinations thereof. Input device 1132 may be interfaced to bus 1112 via any of a variety of interfaces (not shown) including, but not limited to, a serial interface, a parallel interface, a game port, a USB interface, a FIREWIRE interface, a direct interface to bus 1112, and any combinations thereof. Input device 1132 may include a touch screen interface that may be a part of or separate from display 1136, discussed further below. Input device 1132 may be utilized as a user selection device for selecting one or more graphical representations in a graphical interface as described above.

A user may also input commands and/or other information to computer system 1100 via storage device 1124 (e.g., a removable disk drive, a flash drive, etc.) and/or network interface device 1140. A network interface device, such as network interface device 1140, may be utilized for connecting computer system 1100 to one or more of a variety of networks, such as network 1144, and one or more remote devices 1148 connected thereto. Examples of a network interface device include, but are not limited to, a network interface card (e.g., a mobile network interface card, a LAN card), a modem, and any combination thereof. Examples of a network include, but are not limited to, a wide area network (e.g., the Internet, an enterprise network), a local area network (e.g., a network associated with an office, a building, a campus or other relatively small geographic space), a telephone network, a data network associated with a telephone/voice provider (e.g., a mobile communications provider data and/or voice network), a direct connection between two computing devices, and any combinations thereof. A network, such as network 1144, may employ a wired and/or a wireless mode of communication. In general, any network topology may be used. Information (e.g., data, software 1120, etc.) may be communicated to and/or from computer system 1100 via network interface device 1140.

Computer system 1100 may further include a video display adapter 1152 for communicating a displayable image to a display device, such as display 1136. Examples of a display device include, but are not limited to, a liquid crystal display (LCD), a cathode ray tube (CRT), a plasma display, a light emitting diode (LED) display, and any combinations thereof. Display adapter 1152 and display 1136 may be utilized in combination with processor 1104 to provide graphical representations of aspects of the present disclosure. In addition to a display device, computer system 1100 may include one or more other peripheral output devices including, but not limited to, an audio speaker, a printer, and any combinations thereof. Such peripheral output devices may be connected to bus 1112 via a peripheral interface 1156. Examples of a peripheral interface include, but are not limited to, a serial port, a USB connection, a FIREWIRE connection, a parallel connection, and any combinations thereof.

The foregoing has been a detailed description of illustrative embodiments of the invention. Various modifications and additions can be made without departing from the spirit and scope of this invention. Features of each of the various embodiments described above may be combined with features of other described embodiments as appropriate in order to provide a multiplicity of feature combinations in associated new embodiments. Furthermore, while the foregoing describes a number of separate embodiments, what has been described herein is merely illustrative of the application of the principles of the present invention. Additionally, although particular methods herein may be illustrated and/or described as being performed in a specific order, the ordering is highly variable within ordinary skill to achieve methods according to the present disclosure. Accordingly, this description is meant to be taken only by way of example, and not to otherwise limit the scope of this invention.

Exemplary embodiments have been disclosed above and illustrated in the accompanying drawings. It will be understood by those skilled in the art that various changes, omissions and additions may be made to that which is specifically disclosed herein without departing from the spirit and scope of the present invention.

1. An apparatus for filtering a potential signal using catheter velocity during electro-anatomical mapping comprising:
at least a catheter configured for intracardiac use;
at least a localization system configured to detect at least a position signal as a function of a location of the at least a catheter;
at least a computing device, wherein the computing device comprises:
a memory; and
at least a processor communicatively connected to the memory, wherein the memory contains instructions configuring the at least a processor to:
derive at least a velocity signal from the at least a position signal;
identify, using the at least a velocity signal, a local minimum velocity and corresponding:
at least a minimum velocity time; and
at least a minimum velocity position; and
process, using a motion filter, the at least a position signal as a function of the at least a minimum velocity time and the at least a minimum velocity position.

2. The apparatus of claim 1, wherein the at least a catheter comprises at least a transducer configured to detect a cardiac phenomenon and output at least a potential signal, as a function of a cardiac phenomenon.

3. The apparatus of claim 1, wherein the processor is further configured to receive the at least a potential signal from the at least a transducer and the at least a position signal from the at least a localization system, wherein the at least a position signal comprises a cardiac motion and a respiratory motion, wherein the cardiac motion comprises a systole phase and a diastole phase and the respiratory motion comprises an inspiration phase and an expiration phase.

4. The apparatus of claim 3, wherein identifying a local minimum velocity of the catheter for the cardiac motion comprises:
calculating a first derivative of the at least a velocity signal;
determining a threshold to filter discrete points of the at least a velocity signal; and
comparing a first discrete point to a second discrete point.

5. The apparatus of claim 4, wherein removing the cardiac motion and the respiratory motion from the at least a position signal comprises correlating the cardiac motion and the respiratory motion to determine a total motion of the position signal.

6. The apparatus of claim 1, further comprises discretizing, using the at least a processor, the at least a potential signal and the at least a position signal.

7. The apparatus of claim 6, wherein discretizing comprises:
selecting a sample rate for sampling a plurality of discrete points from the at least a potential signal and the at least a position signal; and
quantizing the plurality of discrete points.

8. The apparatus of claim 1, wherein processing, using the motion filter, comprises interpolating the at least a position signal by:
sampling a plurality of discrete points from the at least a position signal;
averaging an intermediate point from a first discrete point and a second discrete point of the plurality of discrete points; and
fitting a function to the sampled discrete points to generate a continuous position signal.

9. The apparatus of claim 8, wherein the continuous position signal is displayed using a display device.

10. The apparatus of claim 1, wherein the at least a localization system comprises one or more of an electromagnetic localization system, an ultrasound-based localization system, an optical localization system, and an impedance-based localization system.

11. A method for filtering a potential signal using catheter velocity during electro-anatomical mapping comprising:
deriving at least a velocity signal from the at least a position signal;
identifying, using the at least a velocity signal, a local minimum velocity and corresponding:
at least a minimum velocity time; and
at least a minimum velocity position; and
processing, using a motion filter, the at least a position signal as a function of the at least a minimum velocity time and the at least a minimum velocity position.

12. The method of claim 11, wherein the at least a catheter comprises at least a transducer configured to detect a cardiac phenomenon and output at least a potential signal, as a function of a cardiac phenomenon.

13. The method of claim 11, wherein the at least a processor is further configured to receive the at least a potential signal from the at least a transducer and the at least a position signal from the at least a localization system, wherein the at least a position signal comprises a cardiac motion and a respiratory motion, wherein the cardiac motion comprises a systole phase and a diastole phase and the respiratory motion comprises an inspiration phase and an expiration phase.

14. The method of claim 13, wherein identifying a local minimum velocity of the catheter for the cardiac motion comprises:
calculating a first derivative of the at least a velocity signal;
determining a threshold to filter discrete points of the at least a velocity signal; and
comparing a first discrete point to a second discrete point.

15. The method of claim 13, wherein removing the cardiac motion and the respiratory motion from the at least a position signal comprises correlating the cardiac motion and the respiratory motion to determine a total motion of the position signal.

16. The method of claim 11, further comprises discretizing, using the at least a processor, the at least a potential signal and the at least a position signal.

17. The method of claim 16, wherein discretizing comprises:
selecting a sample rate for sampling a plurality of discrete points from the at least a potential signal and the at least a position signal; and
quantizing the plurality of discrete points.

18. The method of claim 11, wherein processing, using the motion filter, comprises interpolating the at least a position signal by:
sampling a plurality of discrete points from the at least a position signal;
averaging an intermediate point from a first discrete point and a second discrete point of the plurality of discrete points; and
fitting a function to the sampled discrete points to generate a continuous position signal.

19. The method of claim 18, wherein the continuous position signal is displayed, using a display device.

20. The method of claim 11, wherein the at least a localization system comprises one or more of an electromagnetic localization system, an ultrasound-based localization system, an optical localization system, and an impedance-based localization system.
